(19) United States
(12) Patent Application Publication
(10) Pub. No.: US 2025/0228447 A1
(43) Pub. Date: Jul. 17, 2025

(54) Cleaning Apparatus for Cleaning Internal Channels of an Item of Equipment

(71) Applicant: MEDITECH ENDOSCOPY LTD, Chesterfield (GB)

(72) Inventor: Peter RAMSEY, Chesterfield (GB)

(21) Appl. No.: 18/854,741

(22) PCT Filed: Apr. 5, 2023
(86) PCT No.: PCT/GB2023/050913

§ 371 Date: Oct. 7, 2024

(30) Foreign Application Priority Data
Apr. 6, 2022 (GB) 2205049.6

(51) Int. Cl. A61B 1/12; B08B 3/04; B65B 3/04; B65B 51/10; B65D 75/00; B65D 81/32

(52) U.S. Cl. CPC A61B 1/123; B65B 3/04; B65B 51/10; B65D 75/008; B65D 81/3272; B08B 3/04

(57) ABSTRACT

Cleaning Apparatus for Cleaning Internal Channels of an Item of Equipment A cleaning apparatus for cleaning internal channels of an item of equipment comprises a bag including a main body portion and a neck portion, the main body portion providing an internal volume for holding a first liquid, and a reservoir having an internal volume for holding a second liquid, the reservoir being engaged with the neck portion of the bag such that, in use, when the neck portion is opened to access the internal volume of the main body portion, the second liquid is concurrently released from the reservoir to mix with the first liquid.

### FIELD OF THE INVENTION

The present invention relates to a cleaning apparatus for flushing and cleaning the internal channels of an item of equipment such as an endoscope or similar instrument. This invention also relates to a method of cleaning internal channels of a piece of equipment such as an air/water channel of an endoscope.

### BACKGROUND TO THE INVENTION

FIG. 1 illustrates an air/water system of an endoscope 902. A first component of such an endoscope air/water system is an air pump 904 in a light source 906. The air flows into the endoscope 902 through a light guide connector 908 of the endoscope 902. The air travels through a very small diameter channel 910 in the endoscope 902 and flows out of an air/water valve 912. The air/water system is controlled from a control section 918 of the endoscope 902. During use of the endoscope 902, in order to insufflate an organ to be examined, the air/water valve 912 is gently covered thereby preventing air exiting through the air/water valve 912 and instead diverting air down the insertion tube 920 and out of a distal tip 922 of the endoscope 902.

Another component of the air/water system is a water bottle. The water bottle is attached to air and water ports 914, 916 of the endoscope 902. During use, the air/water valve 912 may be depressed so that air entering the endoscope 902 is diverted through the air port 914 of the endoscope 902 and into the water bottle causing the bottle to be pressurized. This pressure then forces water from the bottle, through the water port 916, and into another small diameter channel 924 in the endoscope. The water travels along the channel 924 and may be used to flush or clear the view at the distal tip 922 of the endoscope 902.

The air/water channel is therefore actually formed by the two separate small diameter channels 910, 924 that combine into a single channel only proximate the distal tip 922 of the endoscope 902. Furthermore, a small metal nozzle is generally disposed at the tip 922 of the endoscope 902 to deflect air or water from the air/water channel 910, 924 across the image lens of the endoscope 902. The presence of this metal nozzle means that it is not possible to brush the air/water channel 910, 924. This, together with the small diameter of the air/water channel 910, 924, as well as other factors, makes it difficult to clean and decontaminate the air/water channel 910, 924.

The air/water nozzle is the area of greatest failure in the channel system of endoscopes as the air/water nozzle is the point of smallest internal diameter of the air/water channel system, which means that it can easily become blocked or obstructed by debris. Furthermore, during an endoscopic procedure, air infused into the organ being examined can force debris to back up into the nozzle and into the air/water channels 910, 924. This debris can travel some distance back into the channels.

The endoscope also includes a suction/biopsy channel 926 that extends to the tip 922 of the endoscope 902. It will be appreciated that it is very important to thoroughly clean and sterilise this suction/biopsy channel 926 to prevent cross-contamination.

Accordingly, after use of an endoscope, the endoscope must be cleaned and sterilised. Cleaning refers to the removal of visible debris, which may include both inorganic and organic substances. Cleaning usually involves a mechanical process, together with water and detergent. Following cleaning, sterilisation of the endoscope destroys microorganisms present on or in the endoscope. Sterilisation processes usually involve steam or a chemical sterilant. Following sterilisation, the endoscope undergoes high-level disinfection.

It is currently recommended that a preliminary cleaning routine should be undertaken immediately after use and before the endoscope is detached from the light source/video processor. Current pre-clean practice is to flush air and water through both the air/water channel and the suction/biopsy channel. It has been found, however, that routine pre-cleaning procedures for endoscopes do not remove biofilm reliably from endoscope channels. This leads to failures in the subsequent decontamination processes.

It is also recommended that the air and water channels, suction/biopsy channel, and any auxiliary channel, are also irrigated with detergent, not only to check for blockages but also to expel any blood, mucus and other debris.

It is an object of the present invention to provide a cleaning apparatus that permits improved cleaning of internal channels of an endoscope, in particular with a detergent solution, and that overcomes at least one disadvantage of prior art endoscope channel flushing or cleaning systems whether referred to herein or otherwise.

### SUMMARY OF THE INVENTION

A first aspect of the invention provides a cleaning apparatus for cleaning internal channels of an item of equipment, the apparatus comprising:
a bag including a main body portion and a neck portion, the main body portion providing an internal volume for holding a first liquid; and
a reservoir having an internal volume for holding a second liquid, the reservoir being engaged with the neck portion of the bag such that, in use, when the neck portion is opened to access the internal volume of the main body portion, the second liquid is concurrently released from the reservoir to mix with the first liquid.

Preferably the internal volume of the reservoir is no more than 10% of the internal volume of the main body portion. In preferred embodiments the internal volume of the reservoir holds 5 ml of liquid and the internal volume of the main body portion holds 500 ml of liquid.

In some embodiments the reservoir comprises a pouch that is suspended from a part of the neck portion of the bag.

In some embodiments the neck portion of the bag is closed at a top furthest from the main body portion by a heat welded seal.

In other embodiments the neck portion of the bag includes a frangible seal that forms a boundary of the reservoir. The frangible seal preferably includes a weld line having an arc-shaped portion forming a concavity for receiving the second liquid. In preferred embodiments the neck portion of the bag is closed at a top furthest from the main body portion by a heat welded seal, such that the reservoir is defined between the frangible seal and the heat welded seal.

The neck portion of the bag may comprise a retainer arranged to engage with said item of equipment to hold a part of said item of equipment in said first liquid.

In preferred embodiments the cleaning apparatus comprises a volume of the first liquid in the internal volume of the main body portion and a volume of the second liquid in the internal volume of the reservoir. The first liquid may be sterile water or distilled water and the second liquid may be a detergent.

A second aspect of the invention provides a method of manufacturing a cleaning apparatus, the apparatus being according to the first aspect of the invention and including a frangible seal that forms a boundary of the reservoir in the neck portion of the bag, and a volume of the first liquid in the internal volume of the main body portion and a volume of the second liquid in the internal volume of the reservoir. The method comprises:
filling the main body portion of the bag with a first volume of the first liquid;
forming a frangible seal across the neck portion of the bag to provide a fluid boundary between the main body portion of the bag and a least an upper region of the neck portion;
filling a part of the neck portion with a second volume of the second liquid; and
sealing a top of the neck portion furthest from the main body portion, such that the second liquid is in a sealed volume between the fragible seal and the top of the neck portion.

Preferably the top of the neck portion is sealed by heat welding.

In preferred embodiments the frangible seal comprises a weld line that extends in a direction generally across the neck portion with a central region of the weld line having a semi-circular shape that extends generally in a direction towards the main body portion and defines a recess of the frangible seal, and the method comprises filling the recess with the second liquid.

Preferably the first liquid is sterile water or distilled water and the second liquid is a concentrated detergent.

A third aspect of the invention provides a method of using a cleaning apparatus, the cleaning apparatus being according to the first aspect of the invention and including a frangible seal that forms a boundary of the reservoir in the neck portion of the bag, and a volume of the first liquid in the internal volume of the main body portion and a volume of the second liquid in the internal volume of the reservoir. The method comprises:
breaking the frangible seal to dispense the volume of second liquid into the volume of first liquid in the main body portion of the bag; and
inserting a piece of equipment through the neck portion of the bag into the liquid in the main body portion of the bag.

In preferred embodiments the neck portion of the bag is closed at a top furthest from the main body portion by a heat welded seal, and the method further comprises breaking the heat welded seal or separating the heat welded seal from the bag.

In preferred embodiments the piece of equipment is an endoscope and the method comprises inserting a tip of the endoscope into the liquid in the main body portion of the bag.

The neck portion of the bag may comprise a retainer and the method preferably comprises engaging the retainer with the item of equipment to retain a part of the item of equipment in the first liquid.

Preferred and/or optional features of each aspect and embodiment described above may also be used, alone or in appropriate combination, in the other aspects and embodiments also.

### BRIEF DESCRIPTION OF THE DRAWINGS

The invention will now be further described by way of example only and with reference to the accompanying drawings, in which like reference signs are used for like features, and in which:

FIG. 1 illustrates an air/water system of an endoscope;

FIGS. 2 to 4 illustrate a cleaning apparatus in an initial state before use according to an embodiment of the present invention, the cleaning apparatus comprising a bag for holding a volume of fluid;

FIGS. 5 to 7 illustrate the cleaning apparatus of FIGS. 2 to 4 in a second state in use, with a tip of an endoscope disposed within the internal volume of the bag;

FIGS. 8 to 13 show the steps in a method of manufacturing a cleaning apparatus according to a further embodiment of the present invention, the cleaning apparatus comprising a bag for holding a volume of fluid;

FIGS. 14 and 15 show the cleaning apparatus of FIGS. 8 to 13 in an initial state before use; and

FIGS. 16 to 19 show the steps in a method of using the cleaning apparatus of FIGS. 8 to 13.

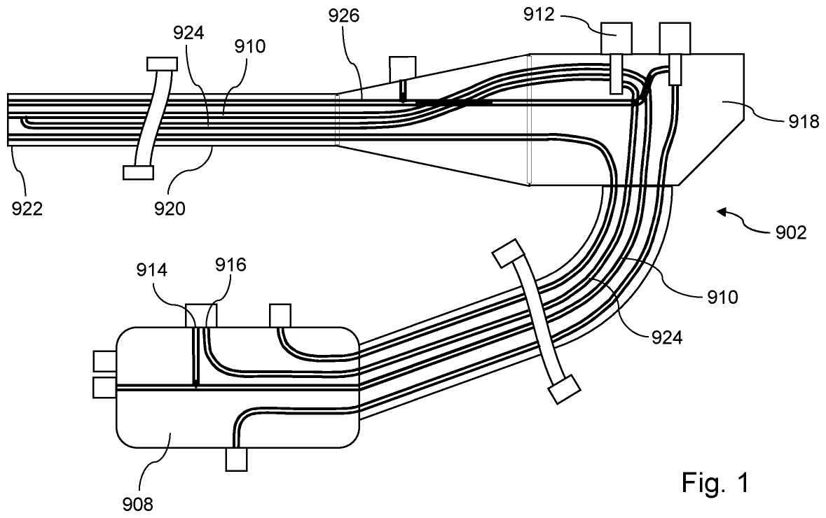

### DESCRIPTION OF THE PREFERRED EMBODIMENTS

The present invention provides a cleaning apparatus that comprises a bag or container for holding a volume of a liquid, which is preferably a detergent solution. In use, a tip or part of a piece of equipment, such as a tip of an endoscope, may be inserted into the internal volume of the container and is preferably retained in a position such that the tip or part of the piece of equipment is held in the volume of liquid. The liquid may be used to clean external surfaces of the piece of equipment. Additionally or alternatively, the liquid may be drawn up through an internal channel of the equipment to flush or clean the channel of the equipment.

When used to clean medical equipment, such as an endoscope or similar, the bag may contain a neutral pH enzymatic detergent solution. The detergent is designed to break down proteinaceous materials and prevent the formation of biofilm, for example in the air/water channel of the endoscope.

For most uses, the detergent solution in the bag is preferably of a relatively low concentration. It has been found, however, that pre-mixed low concentration detergent degrades over time. Tests show that bacterial growth occurs in low concentration detergent. Accordingly, if the cleaning apparatus is stored for a long time before use, the detergent solution in the cleaning apparatus may have degraded to a point at which the cleaning apparatus is no longer fit for purpose.

Accordingly, it is desirable if the low concentration detergent solution is used soon after it is formed. One option is to separate, within the container or bag, a volume of sterile water and a volume of concentrated detergent. These two volumes could then be mixed to create the required low concentration detergent solution shortly before using the cleaning apparatus.

The cleaning apparatus is therefore advantageously provided with a reservoir within the internal volume of the bag. The bag preferably includes a main body portion that provides an internal volume for holding a first liquid. The reservoir preferably has an internal volume for holding a second liquid. In preferred embodiments the first liquid is sterile water and the second liquid is a concentrated detergent.

In an initial state the reservoir holds the second liquid separated from the first liquid. Before use of the cleaning apparatus, the reservoir is preferably ruptured, broken or opened to permit the second liquid to mix with the first liquid. Accordingly, in embodiments in which the first liquid is sterile water and the second liquid is a concentrated detergent, these may be mixed shortly before use to form the lower concentration detergent solution within the internal volume of the bag.

In some embodiments the bag comprises an upper neck portion providing access to the main body portion of the bag and the reservoir is engaged with the neck portion of the bag. In preferred embodiments the reservoir is configured such that, in use, when the neck portion is opened to access the internal volume of the main body portion, the reservoir is concurrently opened such that the second liquid is released from the reservoir to mix with the first liquid.

The internal volume of the reservoir is preferably significantly smaller than the internal volume of the main body portion. The internal volume of the reservoir may be smaller than 20% of the internal volume of the bag. The internal volume of the reservoir may be smaller than 10% of the internal volume of the bag. The internal volume of the reservoir may hold 5 ml of liquid and the internal volume of the bag may hold 500 ml of liquid.

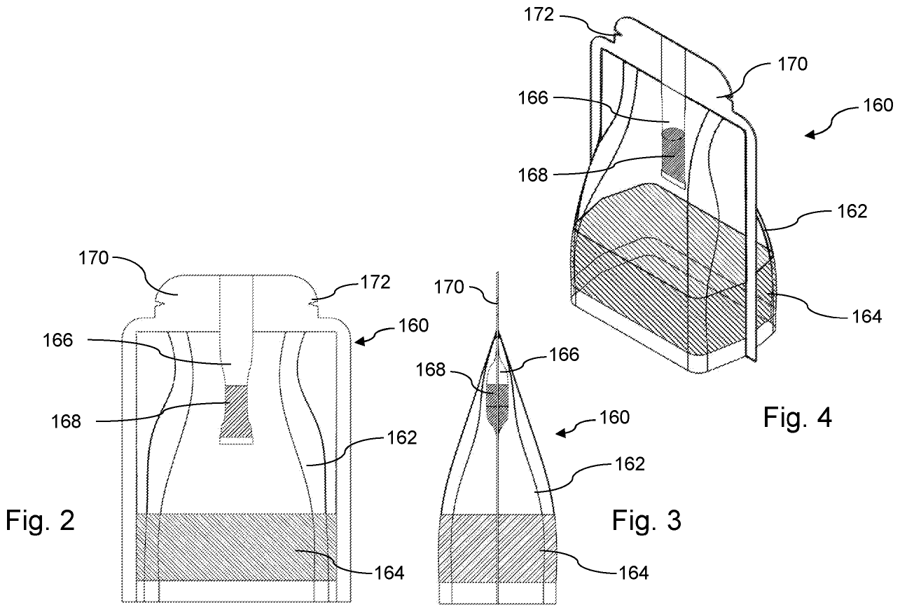

FIGS. 2 to 7 illustrate a cleaning apparatus according to a preferred embodiment of the invention. The cleaning apparatus may, in particular, provide a pre-clean apparatus 160 that can be used to provide an initial cleaning of an endoscope 2.

The apparatus 160 comprises a bag 162 including a main body portion containing a volume of a first liquid, which in this example is sterile water 164. The apparatus further comprises a pouch 166 containing a volume of a second liquid, which in this example is a concentrated detergent 168. The bag 162 includes a neck portion 170 that, when open, provides access to an internal volume of the main body portion. The neck portion is preferably of suitable dimensions to allow a tip or insertion portion of an endoscope 2 to be inserted through the neck 170 and into the main body portion of the bag 162.

In this embodiment the neck 170 of the bag 162 is closed at the top with a heat welded seal. The neck 170 may have two V-groove regions 172 on either side, proximate the top of the neck 170, which will allow a user to tear the top off the neck 170 to open the bag 162. It will be understood that the V-groove regions may provide a guide to a user as to where to tear the neck portion and may provide a point of weakness to assist a user in tearing the neck portion.

The reservoir of this embodiment in is the form of a narrow tubular bag or pouch 166, which is initially separate from the bag. This tubular pouch 166 preferably has a diameter small enough to sit inside the neck 170 of the larger bag 162. During manufacture of the cleaning apparatus, the top of the smaller tubular pouch 166 (reservoir) is preferably heat sealed at its top concurrently with a top of the neck 170 of the larger bag 162. Heat sealing may therefore be used to seal both the reservoir and the bag, and may also attach the reservoir to the bag. In this embodiment the reservoir pouch is attached to the bag so that the reservoir is suspended from a top edge of the neck portion of the bag and hangs down or extends into the main body portion of the bag. In this way, the concentrated detergent 168 is contained within the reservoir 166 in the neck 170 of the larger bag 162 isolated from the sterile water 164 within the main internal volume of the bag 162.

The heat seal is preferably disposed above a line across which a user is guided to tear or cut open the bag to gain access to the internal volume of the bag. Accordingly, when the user tears open the neck 170 of the larger bag 162, the smaller reservoir 166 (now detached from the bag) falls into the main body portion of the larger bag 162. The second liquid (concentrated detergent) 168 is then mixed with the first liquid (sterile water) 164. As such, the lower concentration detergent solution is only formed when a user is ready to use the cleaning apparatus.

This configuration of the reservoir and bag therefore provides a low cost apparatus 160, not requiring additional mouldings.

In preferred embodiments, to allow the cleaning apparatus to be used with medical equipment such as an endoscope, the neck 170 of the bag 162 preferably has a large enough diameter to allow a tip portion or insertion portion of an endoscope 2 to be easily inserted into the main body portion of the bag.

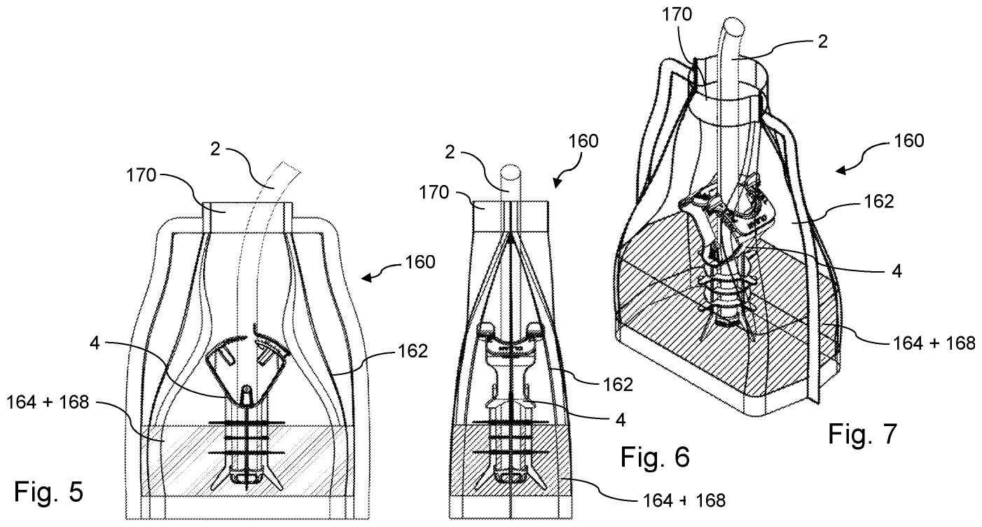

In some cases an endoscope may be fitted with a tip protector 4 (such as that disclosed in EP3206555), as illustrated in FIGS. 5 to 7. Accordingly, it may be preferable for the neck of the bag to have a diameter large enough to allow the tip portion of the endoscope with the tip protector attached to be inserted into the bag 162 and removed from the bag 162 through the neck 170 without the tip protector 4 being pulled off the end of the endoscope 2.

In some applications, a cylindrical sponge is used to wipe detergent onto the endoscope, in particular onto external surfaces of the endoscope. In some examples the cylindrical sponge has a hole through the centre and a slit in one side and is designed to fit around the endoscope insertion tube. In use, a user slides the sponge along the endoscope insertion tube and agitates it back and forth to clean the endoscope. In these cases, the neck of the bag preferably has dimensions large enough to accommodate this sponge so that a user may use a detergent solution within the bag to clean external surfaces of the endoscope. Typically, this type of sponge may be 50 mm in diameter and 100 mm long.

In preferred embodiments, and in particular for use in flushing and cleaning endoscopes, the bag contains 500 ml of sterile water and the reservoir contains 5 ml of concentrated detergent.

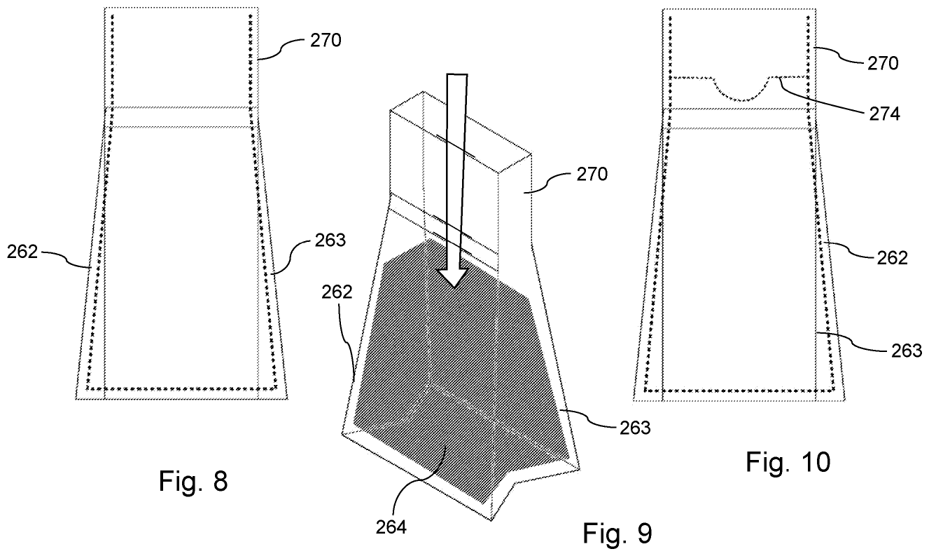

FIGS. 8 to 19 illustrate an alternative example of a pre-clean apparatus 260.

FIGS. 8 to 13 illustrate the features of the cleaning apparatus 260 with reference to method steps involved in manufacturing the cleaning apparatus 260. In this embodiment the cleaning apparatus comprises a bag 262 including a main body portion 263 and a neck portion 270 extending upwardly from the main body portion 263. Sides and a base of the bag are sealed (as indicated by dotted lines in FIG. 8) to form an internal volume of the bag 262. Initially an upper end of the neck portion 270 is open to allow the bag 262 to be filled with a first volume of liquid, as illustrated in FIG. 9. This first volume of liquid is preferably sterile or distilled water 264.

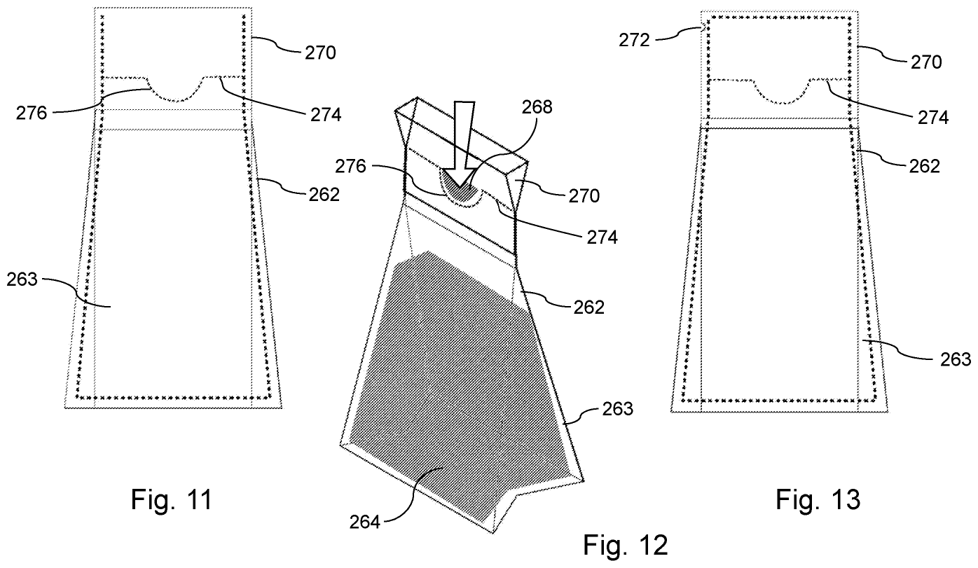

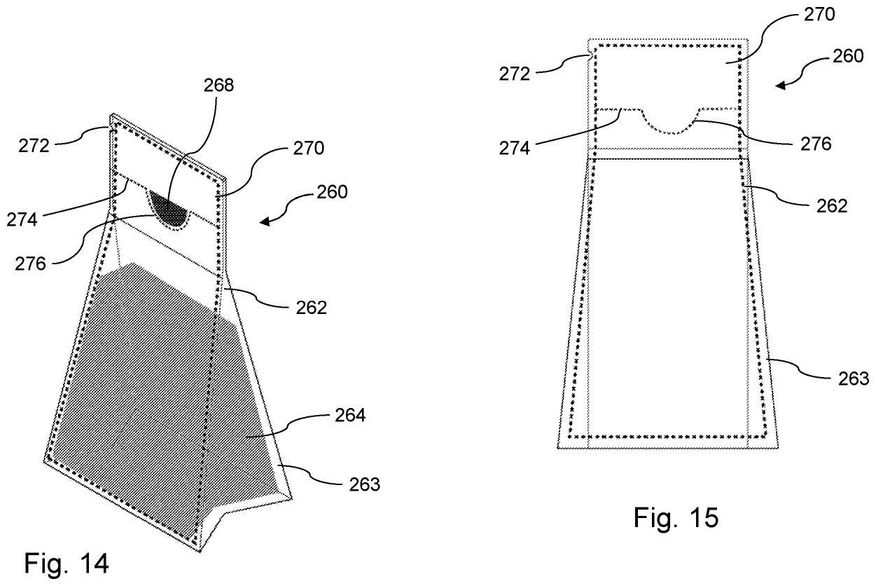

After the first liquid 264 has been poured into the bag 262, a shaped frangible seal 274 is welded across the neck portion 270, as shown in FIG. 10. This frangible seal 274 seals the main body portion 263 of the bag 262 to contain the first liquid (sterile water) 264 within the bag 262. The shape of the frangible seal 274, as best shown in FIG. 11 includes a recess or concavity 276 for receiving a volume of a second liquid, which may be concentrated detergent. To form the recess 276, in this embodiment the frangible seal 274 comprises a weld line that extends in a direction generally across the neck portion 270, with a central region of the weld line having a semi-circular shape that extends generally in a direction towards the base of the bag 262. In a next step, this recess 276 is filled with concentrated detergent 268, as shown in FIG. 12. The upper end of the neck portion 270 is then sealed, as shown in FIG. 13. The seal closing the upper end of the neck portion 270 retains the second liquid (concentrated detergent) in a region of the neck portion between the upper seal and the frangible seal 274. The arrangement of the seals therefore means that the concentrated detergent (second liquid) 268 is held in the neck portion 270 of the bag 262 separated from the sterile water (first liquid) 264 in the main body 263 of the bag 262, as shown in FIG. 14.

A tear slot or cut-out 272 may be provided at one side of the neck portion 270 proximate its upper end. This provides a weakened area of weld to allow a user to subsequently tear open the neck portion 270 of the bag 262 at that point. It will be understood that the tear slot 272 is disposed below the upper seal but above the frangible seal.

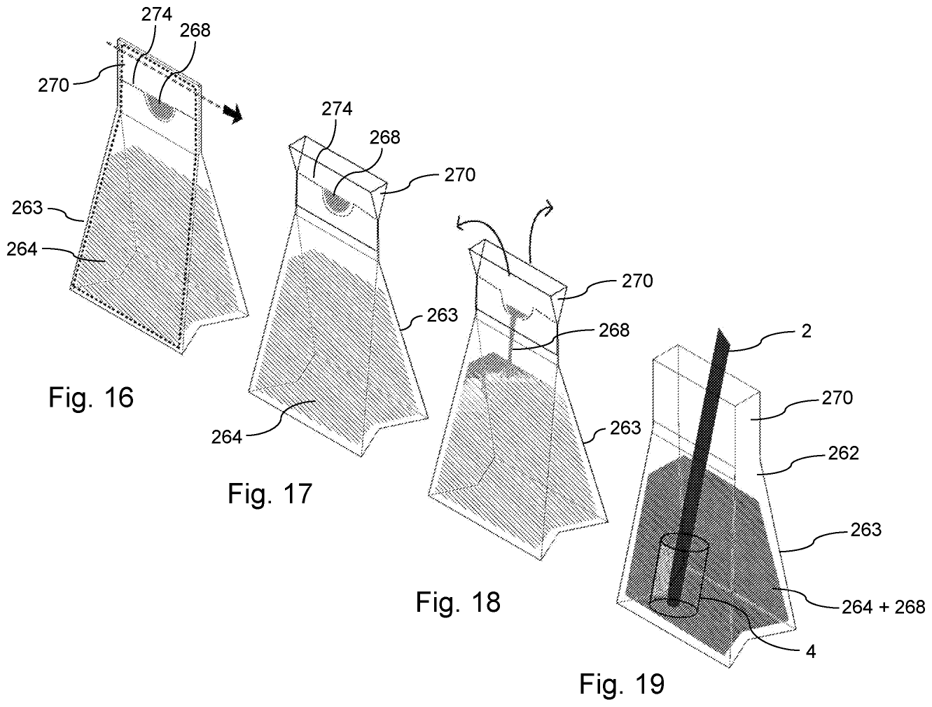

To use the pre-clean apparatus 260, a user first tears or cuts open the upper end of the neck portion 270, below the upper seal and above the frangible seal, as shown in FIG. 16. Subsequently, by pulling on the sides of the neck portion 270 at its upper end, a user can break the frangible seal 274 without tearing the walls of the bag 262. This dispenses the concentrated detergent 268 into the sterile water 264 as shown in FIG. 18. The detergent mixes with the water in the main body 263 of the bag 262.

An endoscope 2 may then be inserted into the main body portion 263 of the bag 262 through the neck portion 270, as illustrated in FIG. 19. A tip section of the endoscope 2 may be cleaned with the detergent solution within the bag 262, with optional use of a suitable sponge as described above. The detergent solution may also be drawn up through the channels of the endoscope to perform a cleaning flush of the channels of the endoscope, in particular a suction/biopsy channel of the endoscope.

In preferred embodiments, the neck portion of the bag includes a retainer for holding the tip of the instrument or piece of equipment within the volume of liquid in the bag during use. The retainer may be in the form of an elastic or sprung clip that grips a part of the instrument or equipment that extends through the neck portion of the bag. The retainer may, for example, be in the form of an elastically deformable ring engaged with the neck portion of the bag. The deformable ring may be biased into a position in which a narrow opening is provided in the neck portion and the deformable ring must be pressed or stretched to increase a dimension of the opening to allow the instrument or piece of equipment to be inserted through the neck portion. With the force on the deformable ring released the ring tries to return to its original position and grips a part of the instrument or piece of equipment.

Alternatively the retainer may comprise a tie, for example a cable tie or similar. In use the tie may be used to tighten a part of the neck portion around a part of the instrument or piece of equipment. The retainer may comprise a first region of the neck portion that may be adhered to a second region of the neck portion. One or both of the first and second regions may include a layer of adhesive. In use the first and second regions may be adhered to each other to effectively wrap the neck portion around a part of the instrument or piece of equipment. The layer of adhesive may be exposed when a user opens the top of the bag to use the cleaning apparatus.

The retainer maybe integral with or separate from the neck portion of the bag.

As described above, in some applications a cylindrical sponge may be used to wipe detergent onto the endoscope. The cylindrical sponge may have a hole or bore through the centre and a slit in one side such that it fits around the endoscope insertion tube. In these cases, the retainer may be configured to grip or clamp the sponge around the endoscope to retain the tip of the endoscope in the bag. This has the advantage that the sponge provides a layer of protection between the retainer and the insertion tube of the endoscope.

Although in the above embodiment the frangible seal was shaped to provide a recess, it will be appreciated that in other embodiments the frangible seal may extend straight across the neck of the bag, or the frangible seal may include a weld line having any suitable shape to form a sealed boundary of the reservoir to initially separate the first and second liquids.

In the examples illustrated above, the bag is shown as having a generally square or rectangular base. This allows the bag to more easily stand upright on a suitable surface during use. It will be appreciated, however, that the bag may be of any suitable shape. In some examples in which it is beneficial for the bag to be stood upright, the bag may be of any suitable shape and either a lower end of the bag may be placed into a receptacle or dish having a suitable shape, or a perforated plate or dish may be included in the internal volume of the bag. In examples in which a perforated plate or dish is provided, this plate or dish may initially be disposed in a central region of the internal volume of the bag and then a user may push the plate or dish towards a base region of the bag to form a flat base to aid in standing the bag on a surface.

Other modifications and variations not explicitly disclosed above may also be contemplated without departing from the scope of the invention as defined in the appended claims.

1. A cleaning apparatus for cleaning internal channels of an item of equipment, the apparatus comprising:
a bag including a main body portion and a neck portion, the main body portion providing an internal volume for holding a first liquid; and
a reservoir having an internal volume for holding a second liquid, the reservoir being engaged with the neck portion of the bag such that, in use, when the neck portion is opened to access the internal volume of the main body portion, the second liquid is concurrently released from the reservoir to mix with the first liquid.

2. The cleaning apparatus according to claim 1, in which the internal volume of the reservoir is no more than 10% of the internal volume of the main body portion.

3. The cleaning apparatus according to claim 1, in which the internal volume of the reservoir holds 5 ml of liquid and the internal volume of the main body portion holds 500 ml of liquid.

4. The cleaning apparatus according to claim 1, in which the reservoir comprises a pouch that is suspended from a part of the neck portion of the bag.

5. The cleaning apparatus according to any one of Claim 1, in which the neck portion of the bag includes a frangible seal that forms a boundary of the reservoir.

6. The cleaning apparatus according to claim 5, in which the frangible seal includes a weld line having an arc-shaped portion forming a concavity for receiving the second liquid.

7. The cleaning apparatus according to claim 1, in which the neck portion of the bag comprises a retainer arranged to engage with said item of equipment to hold a part of said item of equipment in said first liquid.

8. The cleaning apparatus according to claim 1, comprising a volume of said first liquid in the internal volume of the main body portion and a volume of said second liquid in the internal volume of the reservoir.

9. The cleaning apparatus according to claim 8, in which the first liquid is sterile or distilled water and the second liquid is a detergent.

10. The cleaning apparatus according to claim 8, in which the neck portion of the bag is closed at a top furthest from the main body portion by a heat welded seal.

11. The cleaning apparatus according to claim 5, in which the neck portion of the bag is closed at a top furthest from the main body portion by a heat welded seal, such that the reservoir is defined between the frangible seal and the heat welded seal.

12. A method of manufacturing a cleaning apparatus, the apparatus being according to claim 20, and the method comprising:
filling the main body portion of the bag with a first volume of the first liquid;
forming a frangible seal across the neck portion of the bag to provide a fluid boundary between the main body portion of the bag and a least an upper region of the neck portion;
filling a part of the neck portion with a second volume of the second liquid; and
sealing a top of the neck portion furthest from the main body portion, such that the second liquid is in a sealed volume between the frangible seal and the top of the neck portion.

13. The method of manufacturing a cleaning apparatus according to claim 12, in which the top of the neck portion is sealed by heat welding.

14. The method of manufacturing a cleaning apparatus according to claim 12, in which the frangible seal comprises a weld line that extends in a direction generally across the neck portion with a central region of the weld line having a semi-circular shape that extends generally in a direction towards the main body portion and defines a recess of the frangible seal, and the method comprises filling the recess with the second liquid.

15. The method of manufacturing a cleaning apparatus according to claim 12, in which the first liquid is sterile water or distilled water and the second liquid is a concentrated detergent.

16. A method of using the cleaning apparatus according to claim 20, and the method comprising:
breaking the frangible seal to dispense the volume of said second liquid into the volume of said first liquid in the main body portion of the bag; and
inserting a piece of equipment through the neck portion of the bag into the liquid in the main body portion of the bag.

17. The method of using a cleaning apparatus according to claim 16, in which the neck portion of the bag is closed at a top furthest from the main body portion by a heat welded seal, and the method further comprises breaking the heat welded seal or separating the heat welded seal from the bag.

18. The method of using a cleaning apparatus according to claim 16, in which the piece of equipment is an endoscope and the method comprises inserting a tip of the endoscope into the liquid in the main body portion of the bag.

19. The method of using a cleaning apparatus according to claim 16, in which the neck portion of the bag comprises a retainer and the method comprises engaging the retainer with the item of equipment to retain a part of the item of equipment in the first liquid.

20. The cleaning apparatus according to claim 1, in which the neck portion of the bag includes a frangible seal that forms a boundary of the reservoir, and the cleaning apparatus comprises a volume of said first liquid in the internal volume of the main body portion and a volume of said second liquid in the internal volume of the reservoir.
